(19) United States
(12) Patent Application Publication
(10) Pub. No.: US 2022/0142943 A1
(43) Pub. Date: May 12, 2022

(54) Method of Producing Cannabidiol Derived Products

(71) Applicant: Christine Meeusen, Merced, CA (US)

(72) Inventor: Christine Meeusen, Merced, CA (US)

(21) Appl. No.: 17/579,920

(22) Filed: Jan. 20, 2022

Related U.S. Application Data
(63) Continuation-in-part of application No. 15/842,662, filed on Dec. 14, 2017, now abandoned.
(60) Provisional application No. 62/435,007, filed on Dec. 15, 2016.

(51) Int. Cl. A61K 31/05; A61K 36/185

(52) U.S. Cl. CPC A61K 31/05; A61K 2236/11; A61K 36/185

(57) ABSTRACT

Methods are provided for producing cannabidiol (CBD)-based products in accordance with cycles of the moon and a plurality of biodynamic rituals. In some embodiments, the phase of the moon may be correlated with positions of the moon among the twelve zodiac signs, and the positions used to guide in the cultivation and harvest of cannabis plants. In some embodiments, the phase of the moon and the corresponding astrological phase that the moon is moving through may be used to determine when to plant seeds, when/how to water and fertilize, when to take cuttings, and the right time to harvest mature cannabis plants to produce optimal CBD containing products. Additional methods prior to and during the extraction process may lead to better producing and/or tasting products. It is contemplated that farming cannabis with consideration of moon cycles and various cleansing processes may result in healthier plants, increased yields, and superior products.

### PRIORITY

This application is a continuation-in-part of U.S. patent application Ser. No. 15/842,662, filed Dec. 14, 2017, which claims the benefit of and priority to U.S. Provisional Application No. 62/435,007, filed Dec. 15, 2016, both entitled “Method Of Producing Cannabidiol Containing Products,” and are incorporated by reference herein in their entirety.

### FIELD

The field of the present disclosure generally relates to cannabis-based products. More particularly, the field of the invention relates to a method for producing CBD containing products according to cycles of the moon and a plurality of biodynamic rituals.

### BACKGROUND

In the past 25 years, scientific research into cannabis has demonstrated the existence of naturally occurring compounds and receptors in the brain and body broadly referred to as the endogenous cannabinoid system. More specifically, cannabinoids—neurotransmitters natively produced in the body—have been found to impact regulation of one's mood, pain, memory, appetite, and immune response.

Two of the main active compounds in cannabis are tetrahydrocannabinol (THC) and cannabidiol (CBD), among many others. THC is comparable to the naturally occurring neurotransmitter anandimide (which may also be found in chocolate) and is generally considered to be responsible for marijuana's psychoactive effects. Notably, CBD is non-psychoactive and may have a range of beneficial effects, including as an anti-inflammatory and anti-anxiety agent.

Scientific study has also demonstrated that the endocannabinoid system may have broad impact throughout the body and mind, especially considering that cannabis contains a large range of specific compounds that impact this system, in varying ratios. Accordingly, marijuana's effects on the body may be very subjective, and differ according to the strain and dosage ingested.

Despite harsh laws against its use, recent studies indicate that marijuana is the third most popular recreational drug in America (behind only alcohol and tobacco) and has been used by nearly 100 million Americans. Furthermore, recent polls indicate that a majority of Americans now support legalization of marijuana and support is growing each year. Several states have already decriminalized marijuana, legalized it for medical use, or legalized it completely. As the cultural perception of cannabis becomes less negative, production is bound to increase.

### SUMMARY

Methods are provided for producing CBD-based products in accordance with cycles of the moon and a plurality of biodynamic rituals. In some embodiments, the phase of the moon may be correlated with positions of the moon among the twelve zodiac signs, and the positions used to guide in the cultivation and harvest of cannabis plants. More specifically, the phase and position of the moon relative to Earth, and the corresponding astrological phase that the moon is moving through may be used to determine when to plant seeds, when/how to water and fertilize, when to take cuttings, and the right time to harvest mature CBD-producing plants, such as cannabis plants to produce optimal CBD-containing products. It is contemplated that farming cannabis with consideration of moon cycles may result in healthier plants, increased yields and superior products. The methods described herein may be performed indoors or outdoors without exceeding beyond the spirit and scope of the present disclosure.

In an exemplary embodiment, a method for producing a CBD-containing product comprises determining a moon phase; performing at least one ritual on the first day of a moon cycle; communicating one or more incantations; cleansing each participant of the CBD production; distributing cannabis into one or more containers; extracting CBD from the cannabis; and forming the CBD-containing product.

In another exemplary embodiment, determining the moon phase includes identifying an occurrence of a full moon. In another exemplary embodiment, determining the moon phase includes establishing the moon phase as any one of waning crescent, third quarter, waning Gibbous, full moon, waxing Gibbous, first quarter, and waxing crescent. In another exemplary embodiment, determining the moon phase further comprises identifying positions of the moon among the twelve zodiac signs, and using the positions to guide in the cultivation and harvest of the cannabis plants.

In another exemplary embodiment, performing the at least one ritual includes considering a first zodiac sign prior to the at least one ritual. In another exemplary embodiment, performing the at least one ritual includes performing a biodynamic ritual that comprises lighting bundles of sage. In another exemplary embodiment, performing the at least one ritual includes performing the biodynamic ritual to bless a work environment. In another exemplary embodiment, cleansing comprises lighting a bundle of sage and sweeping the sage over any of a plurality of bottles and ingredients that may include a high-proof alcohol.

In another exemplary embodiment, distributing comprises disposing appropriate dosages of cannabis into one or more preparation jars that are suitable for production of one or more CBD containing products. In another exemplary embodiment, extracting is comprised of a solvent-based extraction method that includes submerging the cannabis in a plurality of suitable solvents to produce a resultant CBD concentrate, and straining the resultant CBD concentrate of plant matter and solvents. In another exemplary embodiment, extracting is comprised of a CO2 extraction method that includes subjecting the cannabis to carbon dioxide under high pressure and very low temperature so as to produce a resultant CBD concentrate, and straining the resultant CBD concentrate of plant matter. In another exemplary embodiment, extracting includes infusing the cannabis with olive oil so as to produce a resultant CBD concentrate.

In an exemplary embodiment, a method for producing CBD-containing products comprises: determining an instance of a new moon; planting cannabis cuttings during a beginning of a first quarter of the lunar cycle; performing at least one biodynamic ritual during a third quarter of the lunar cycle; maintaining cannabis plants during a fourth quarter of the lunar cycle; celebrating rest and reflection through one or more biodynamic rituals during the third quarter and the fourth quarter; and harvesting mature cannabis plants under the glow of the full moon during the third quarter.

In another exemplary embodiment, determining includes correlating the lunar cycle with the twelve zodiac signs so as to guide in the cultivation and harvest of the mature cannabis plants. In another exemplary embodiment, maintaining cannabis plants includes implementing any of watering, fertilizing, and managing pests.

In another exemplary embodiment, the method further comprises extracting a CBD concentrate from harvested mature cannabis plants. In another exemplary embodiment, extracting includes submerging cannabis in one or more suitable solvents and straining of plant matter and solvents to produce the CBD concentrate. In another exemplary embodiment, extracting includes subjecting the cannabis to carbon dioxide under high pressure and very low temperature and straining of plant matter to produce the CBD concentrate. In another exemplary embodiment, extracting includes infusing the cannabis with olive oil and straining of plant matter to produce the CBD concentrate. In another exemplary embodiment, the method further comprises incorporating a suitable quantity of the CBD concentration into any of a plurality of desired CBD-based products.

### BRIEF DESCRIPTION OF THE DRAWINGS

The drawings refer to embodiments of the present disclosure in which:

FIG. 1 illustrates an exemplary method of producing CBD containing products, taking into account moon cycles and a plurality of secular, biodynamic rituals;

FIG. 2 illustrates moon phases for harvesting CBD-producing plants in accordance with various embodiments of the disclosure;

FIG. 3 illustrates zodiac signs that correspond to a plurality of moon phase ranges in accordance with various embodiments of the disclosure;

FIG. 4 illustrates a process 400 for determining an optimal time to plant and process CBD-producing plants in accordance with various embodiments of the disclosure; and

FIG. 5 illustrates a process 500 for producing CBD containing products in accordance with various embodiments of the disclosure.

While the present disclosure is subject to various modifications and alternative forms, specific embodiments thereof have been shown by way of example in the drawings and will herein be described in detail. The invention should be understood to not be limited to the particular forms disclosed, but on the contrary, the intention is to cover all modifications, equivalents, and alternatives falling within the spirit and scope of the present disclosure.

### DETAILED DESCRIPTION

In the following description, numerous specific details are set forth in order to provide a thorough understanding of the present disclosure. It will be apparent, however, to one of ordinary skill in the art that the invention disclosed herein may be practiced without these specific details. In other instances, specific numeric references such as “first element,” may be made. However, the specific numeric reference should not be interpreted as a literal sequential order but rather interpreted that the “first element” is different than a “second element.” Thus, the specific details set forth are merely exemplary. The specific details may be varied from and still be contemplated to be within the spirit and scope of the present disclosure. The term “coupled” is defined as meaning connected either directly to the component or indirectly to the component through another component. Further, as used herein, the terms “about,” “approximately,” or “substantially” for any numerical values or ranges indicate a suitable dimensional tolerance that allows the part or collection of components to function for its intended purpose as described herein.

In general, the present disclosure describes methods for producing CBD containing products according to cycles of the moon and a plurality of biodynamic rituals. More specifically, embodiments of the present disclosure utilize the phase and/or position of the moon relative to Earth and the corresponding astrological phase that the moon is moving through, so as determine when to plant seeds, take cuttings, when/how to water and fertilize, and the right time to harvest plants to produce CBD containing products. It is envisioned that farming marijuana with consideration of moon cycles may result in healthier plants, more potent buds and increased yields. It should be understood that methods described herein may be performed indoors or outdoors without exceeding beyond the spirit and scope of the present disclosure.

As an introductory matter, reference to biodynamic rituals (or ceremonies) herein indicates one or more activities that may be performed so as to promote understanding and appreciation of soil fertility, plant growth, and plant ripeness prior to harvesting. Often, spiritual, astrological and mystical perspectives are emphasized as part of these rituals. As such, it should be understood that references to biodynamic rituals should not be limited to any specific ritual or style thereto. Choosing how to perform the ritual may take into account lunar and astrological influences on soil and plant development. More specifically, as discussed herein, one may choose to plant, cultivate or harvest various crops based on both the phase and/or position of the moon relative to Earth and the zodiacal constellation the moon is passing through. On may also consider, for example, whether the crop growth is at the root, leaf, flower, or fruit of the plant stage.

In one embodiment, a method for producing CBD containing products is disclosed, comprising: determining a moon phase; performing a first biodynamic ritual on the first day of a moon cycle; communicating a first incantation; cleansing each participant of the CBD production; and distributing marijuana in one or more containers. In one embodiment, the moon phase begins on a full moon. In one embodiment, a first zodiac sign is considered prior to performing the first biodynamic ritual. In one embodiment, the first biodynamic ritual comprises lighting bundles of sage. In another embodiment, participants of the biodynamic ritual are cleansed. In one embodiment, the moon phase is any one of: waning crescent, third quarter, waning gibbous, full moon, waxing gibbous, first quarter; and waxing crescent.

With this understanding, it is to be understood that the predictable orbit of the moon may influence the ebb-and-flow of bodies of water around the earth, such as the oceanic tides. Considering that plants are comprised of mostly water, plants can be effected by the moon. Indeed, even the soil in which the plant resides may either retain water in a superior manner or dry out more rapidly, depending on the gravitational pull of the moon. Similarly, seeds may also respond by sprouting sooner and creating more robust plants when the moon is in a particular state, such as a waxing state. Furthermore, it is believed that the effects of the moon's position in the zodiac may be partially explained by differences in electromagnetic currents transmitted by each respective planet.

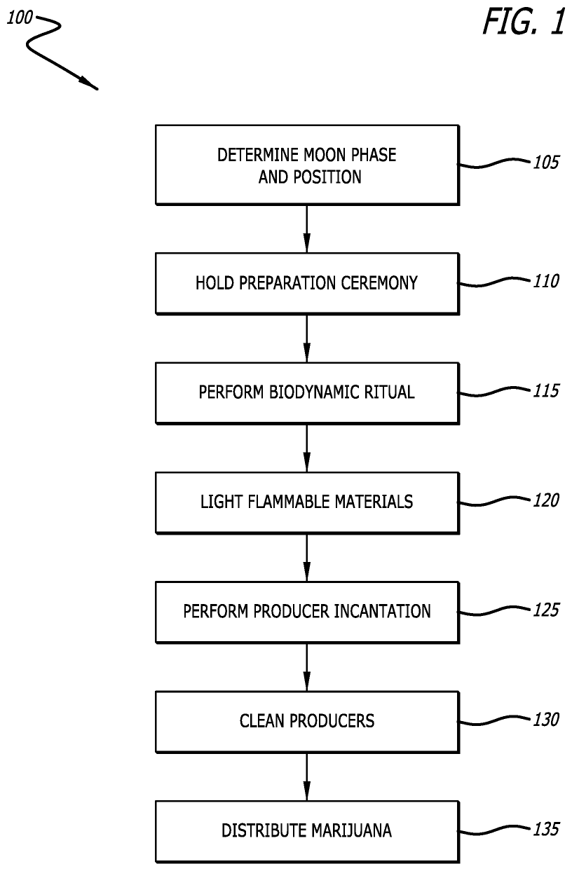

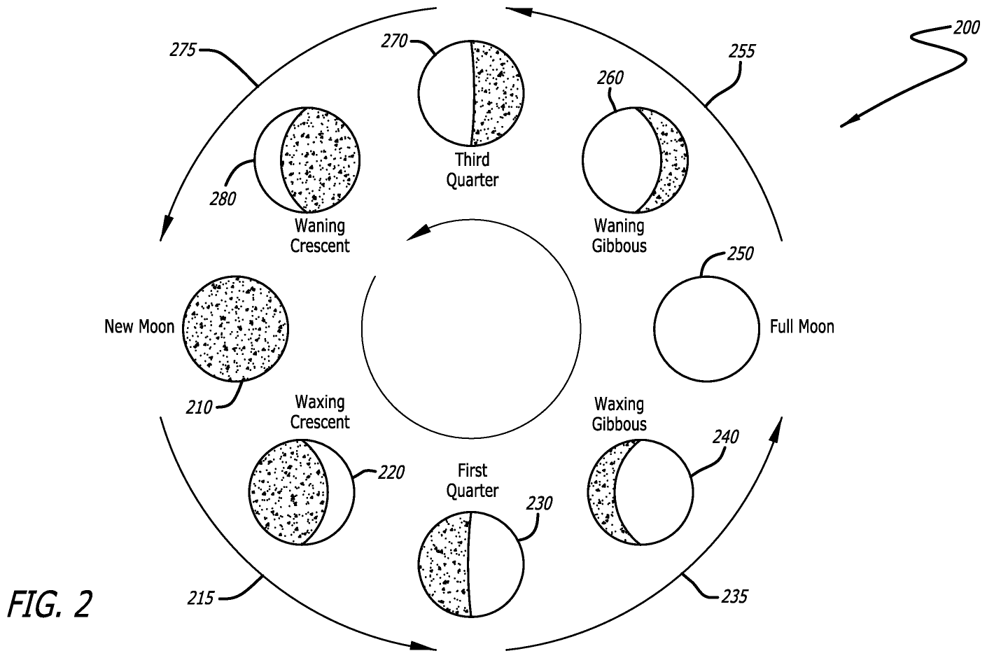

Referring now to the exemplary method 100 of FIG. 1, according to certain embodiments, the moon's phase and position the moon can be determined (block 105). It will be understood that it takes approximately 29.5 days for the phases to complete four stages of the lunar cycle. In one embodiment, a “New Moon” occurs when the moon is roughly in the same direction as the sun. In other words, the moon's illuminated half faces away from the Earth, and therefore the part that faces the Earth is not visible. Accordingly, the sun, Earth and moon align in almost a straight line, with the moon in between the sun and the Earth. Further positions are described in more detail within the discussion of FIG. 2.

In many embodiments, on the first day of the cycle, a biodynamic ritual may be held under the stars so as to prepare/bless a work environment, such as a table (block 110). A biodynamic ritual may subsequently be performed which can include one or more acts that show appreciation with respect to a Creator God and Mother Goddess (block 115). Depending on the product, the biodynamic ritual may be include treating the air with one or more chemicals such as lighting a flammable material on fire such as a bundle of sage which can subsequently be swept over any plurality of bottles and/or ingredients which may include a high-proof alcohol to dissolve CBD-producing plants (block 120). A plurality of incantations may also be communicated which can be designed to help prime one or more producers who tend to the CBD-producing plants (block 125). As described in more detail below, these incantations may be a form of meditation that can help focus the producer before continuing with their work. The producers may clean themselves or each other prior to working with the CBD-producing plants (block 130). In some embodiments, this cleansing may comprise exposing the participants to another airborne chemical as described above.

Finally, in many embodiments, appropriate dosages of marijuana in various forms may be distributed into one or more preparation jars so that one or more CBD containing products may be produced (block 135). In certain embodiments, this distribution will be in one or more extracting solutions and/or containers. This extraction process can take the CBD out of the CBD-producing plant, allowing it to be available in a more useful form once the plant material has been strained from the extraction solution/container. It will be appreciated that to maximize the amount of CBD produced during an extraction, CBD-producing plants, such as cannabis varieties with high CBD producing content are preferred. The type of CBD extraction is crucial to getting good quality CBD extracts. In one embodiment, a solvent-based extraction method may be utilized, wherein a CBD-producing plant, such as a cannabis plant, is submerged in a plurality of solvents. Thereafter, the resulting concentrate may be strained of plant matter and solvents. It should be appreciated that this method requires great caution and precision. In one embodiment, a CO2 extraction method may be implemented, wherein carbon dioxide is subjected to high pressure and very low temperatures so as to produce the concentrate. In one embodiment, olive oil may be infused with a CBD-rich plant so as to produce a CBD concentrate.

Referring now to FIG. 2, moon phases within a lunar cycle 200 for harvesting CBD-producing plants in accordance with various embodiments of the disclosure is shown. The moon orbits the Earth approximately every 29.5 days. The change of the position of the moon in relation to the Earth causes a lunar cycle 200 of visible changes to the moon that we have called moon phases. Broadly, the moon can be understood to start as a new moon 210 that is not illuminated by the sun, and subsequently gets “bigger” as more of it becomes illuminated until it fully illuminated during a full moon 250 approximately halfway during the cycle. The process reverses from the full moon 250, losing illumination until no illumination exists and another new moon 210 is present. This cycle repeats and is predictable.

This lunar cycle 200 can be divided into quarters for easier tracking. A new quarter 215 occurs between the start of the new moon 210 and the moon being hallway illuminated, called the first quarter moon 230. Once the illumination of the moon reaches the first quarter moon 230, the first quarter 235 is considered to begin until a full moon 250 is present. The second third quarter 255 is classified as the time between a full moon 250 and the moon losing half of its illumination until it becomes a third quarter moon 270. Similar to the first quarter moon 230, the third quarter moon 270 occurs when the moon loses half of the available illumination. When the third quarter moon 270 does occur, the start of the third fourth quarter 275 starts until all illumination is lost and a new moon 210 occurs again.

As the moon orbits about the Earth, the illuminated half may be more visible or “growing” from nothing and can be considered a “waxing” stage. At first, only a sliver of the moon is visible, which grows as days go by. This phase is known as a waxing crescent moon 220. Approximately one week after the new moon 210, when the moon has completed about a quarter of its turn around the Earth, half of the illuminated part may be viewed. In other words, a quarter of the moon is visible, and thus it is referred to as the first quarter moon 230. During the next week, more of the illuminated part of the Moon may be seen, and it is now referred to as a waxing gibbous moon 240 (or “humped moon”).

Approximately two weeks after the new moon, the moon is halfway through its revolution. Now, it is to be understood that the illuminated half coincides with the one facing Earth, so that a full disc is substantially visible, which is referred to as a full moon 250. At this time, the moon rises when the sun sets, and sets when the sun rises. If the moon happens to align exactly with the Earth and sun, then a lunar eclipse is established.

Thereafter, until it becomes new again, the illuminated part of the moon decreases, and it is referred to as waning. The first week after the full moon, the moon wanes as a waning gibbous moon 260. Approximately three weeks after the new moon 210 begins, half of the illuminated part of the moon may be viewed again. The illuminated half is opposite that of the first quarter moon 230 and is referred to as a third quarter moon 270 for marking three quarters of the way through the lunar cycle 200. Finally, during about the fourth week of the lunar cycle 200, the moon is reduced to a thin sliver before becoming another new moon 210. This remaining sliver moon is called a waning crescent moon 280.

With regard to the lunar cycle 200, the moon advances through the four quarters 215, 235, 255, 275. The first quarter 215 begins with no visible moon moving through a waxing crescent moon 220 toward a first quarter moon 230. At this stage, moisture is at its peak, so this is generally regarded as a fertile time to take cuttings and to plant in an effort to produce high quality CBD containing products. More importantly, the growth of the roots and the leaves are in balance.

The second quarter 235 begins approximately seven days after the new moon, ending with the full moon 250. From the new moon 210 to the full moon 250 is a period of waxing, characterized by increased, sometimes rapid, growth and fertility, such as the during the waxing crescent 220. The second quarter 255 of the lunar cycle begins with the full moon 250, which, as the name implies, may be considered as a time of completion. In one embodiment, a biodynamic ritual may be performed to celebrate the fruits of the producers and/or harvesters' labor by harvesting under the glow of the full moon. While the full moon 250 brings a peak in moisture levels similar to the new moon, in one embodiment, it is generally not recommended to plant on the full moon 250, as the subsequent waning in light and gravitational pull diminishes growth in the leaves. In one embodiment, during the third quarter 275 of the moon's cycle, energy may be waning, and thus, the growth energy is pulled down into the roots. During this quarter of the lunar cycle 200, planting is not recommended, but general maintenance may be performed. In some embodiments, during this period, plants may be watered, fertilized, and pest management may be implemented. In certain embodiments, a biodynamic ritual may be performed during the third and fourth quarters to celebrate rest and reflection.

Other aspects of the moon and/or astronomical bodies may affect various gravitational pulls that can affect plant growth. In various embodiments, the distance between the moon and the Earth may be utilized to determine an optimal planting time. The moon may vary in distance over time multiple times per year between a minimum and maximum distance. The apex of these distances can lead to a supermoon or micro-moon which may be defined as being within ninety percent of the maximum perigee (closest distance) and apogee (farthest distance) and respectively. Additionally, the apsidal precession of the moon fluctuates over the years as the orbit around the Earth is not directly circular or uniform. This can be from the tilt from the moon's orbit in relation to the Earth. As such, methods and process described herein may be reversed or otherwise altered in response to planting CBD-producing plants in the southern hemisphere of the Earth. All of these changes can be utilized when making a determination when the optimal time to plant or harvest CBD-producing plants should occur.

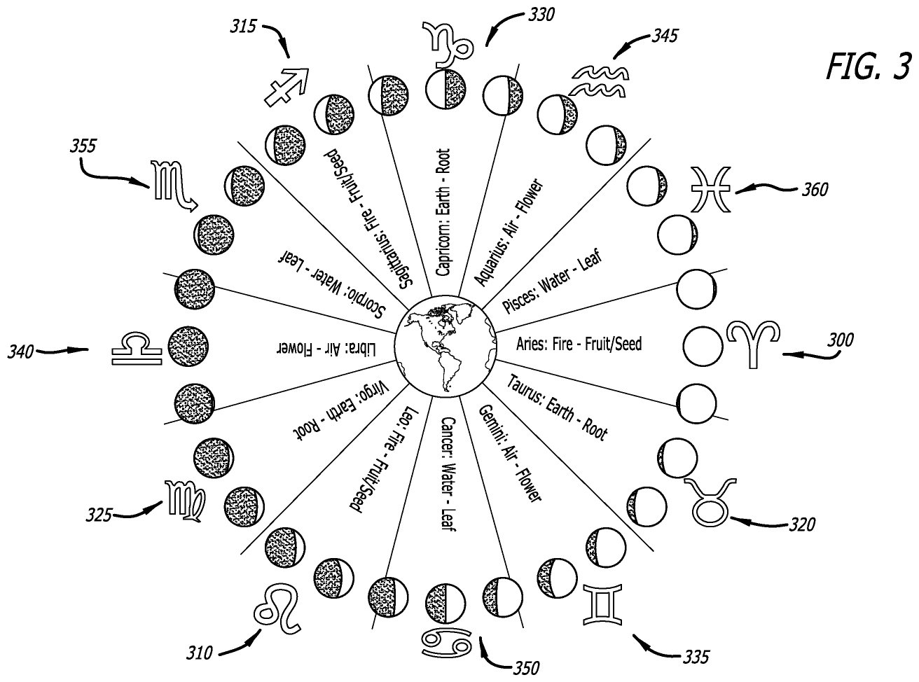

Referring now to FIG. 3, applying the lunar cycle also takes into account the planets and the twelve zodiac signs. In one embodiment, the moon spends approximately two-and-one-third days in each zodiac sign. Accordingly, the moon travels through all twelve zodiac signs substantially every twenty-eight days. It is envisioned that each zodiac sign features qualities of one of each of our four earthly elements: earth, air, fire and water. In one embodiment, each element may also be given masculine or feminine attributes. In one embodiment, dry, hot, barren attributes are considered masculine. In one embodiment, moist, cool, fertile attributes are considered feminine.

Referring still to FIG. 3, Aries 300 is a fire element, featuring one or more masculine attributes. When the moon is in Aries 300, it may be a good time to check plants for bugs, mold or yellowing leaves. However, this is not a recommended time for planting or starting seeds. The same holds true for Leo 310. On the other hand, while Sagittarius 315 is also not a recommended time for planting, it is fine to cultivate and harvest marijuana so as to produce CBD containing products. Moving now to Taurus 320, this zodiac sign features certain feminine qualities as an Earth element. With the lunar movement in this sign, plants are fertile. Accordingly, during orbit through a Taurus 320 sign, plants may respond especially well to planting. On the other hand, while Virgo 325 features moist and feminine Earth qualities, it is a relatively barren time better left to removing pests such as with respect to Aries 300, rather than planting seeds. Capricorn 330, the final earth sign, is more productive than Virgo 325, but it is not generally considered to be as productive as Taurus 320.

Still with respect to FIG. 3, Gemini 335 is dry and barren due to its airy and masculine qualities. During the Gemini 335 phase, transplanting or planting is not recommended, but general maintenance is fine. Similarly, although primarily dry and barren, Libra 340 features enough moisture to encourage fertility and strong root growth, and is thus a good period of time for transplanting. Aquarius 345, while not fertile, may be a good time to harvest the crops. The water signs: Cancer 350, Scorpio 355 and Pisces 360 are all generally considered to be feminine signs. Consequently, these three zodiac signs are understood to be “happy times” for successful growing of marijuana. In some embodiments, when the moon is in Cancer, it may be ideal to take cuttings, plant seeds and transplant as necessary. During Scorpio 355, a productive and abundant time for planting may be observed.

In one embodiment, the moon cycles may be utilized so as to develop any of a plurality of CBD containing products, including but not limited to topical salves, tinctures, and oils made from hemp extract, so as to provide relief from pain, PTSD, debilitating diseases, and more. In one embodiment, the cannabis salve may be used to heal back pain, arthritis and erase scar tissue. The salve may be made from a combination of cannabis trim, coconut oil, vitamin E, lavender oil, calendula oil, and beeswax in varying quantities. In one embodiment, high-CBD cannabis tincture may be turned every morning and night, according to one biodynamic ritual. In one embodiment, the CBD tincture is only bottled during a full moon, after the moon cycle completes. In one embodiment, CBD oil is comprised of 97% pure CBD crystalline. In one embodiment, the CBD products may be organic, lab-tested and pesticide-free, or any combination thereof.

In one embodiment, the CBD containing products are only manufactured from the new moon to the full moon. On the first day of the cycle, a biodynamic ritual may be held under the stars so as to bless a work environment, such as a table. The ritual may include appreciation with respect to a Creator God and Mother Goddess. Depending on the product, a bundle of sage may be lit, and swept over any plurality of bottles and ingredients that include a high-proof alcohol to dissolve the bud. A plurality of incantations may also be communicated. In one embodiment, the producers may utilize a bundle of sage so as to cleanse one another prior to production. In one embodiment, appropriate dosages of marijuana may be scooped into preparation jars.

In one embodiment, a Palo Santo stick may be utilized in one or more biodynamic ritual. It is to be appreciated that the smell of the wood when burned may induce happiness in the preparer. In one embodiment, to brew a fresh batch of salve, in conjunction with a biodynamic ritual, first gloves are worn on the hands, and preparations begin. During a first step of preparation, candles may be lit, sage may be burned, and soft music may be played. In one embodiment, music or chanting indicating that “The earth is our mother, we must take care of her” may be repeated. Prior to distributing the ingredients into a plurality of pots, a prayer huddle may be performed.

During the prayer huddle, if multiple preparers are present, hands may be held and everyone's eyes may be closed. In one embodiment, one of the preparers may indicate appreciation by chanting “Thank you for calling us to do this divine work,” preferably softly and slowly, with conviction. In one embodiment of the biodynamic ritual, one may chant “Let our medicine travel safely and do the work it needs to, and then let the people using our medicine do the work they need to do. Keep our hearts pure and their hearts pure. Keep our minds pure and our hands steady.” Preferably, one or more vows may be taken, including for example, servitude, activism, obedience, ecology, poverty. More specifically, during the two weeks from new moon until full moon, veganism and celibacy may be undertaken.

In one embodiment, pure plant CBD oil may be composed using CO2 extraction methods, so as to comprise substantially 360 mg of CBD per gram of oil. In one embodiment, topical salve may be implemented using the aforementioned techniques so as to assist carrying the CBD compounds into one's system through the skin. In some embodiments, it is envisioned that there will be no difference between the potency of one or more CBD-infused oils and CBD tinctures. It should be appreciated that both CBD-infused oils and CBD tinctures are mediums of getting the medicine into the system via drops on the tongue. In one embodiment, the CBD infused oil is comprised from liquid coconut oil and may have a mild taste. The tincture, however, is comprised of food-grade high-grain alcohol and high-CBD organic plant.

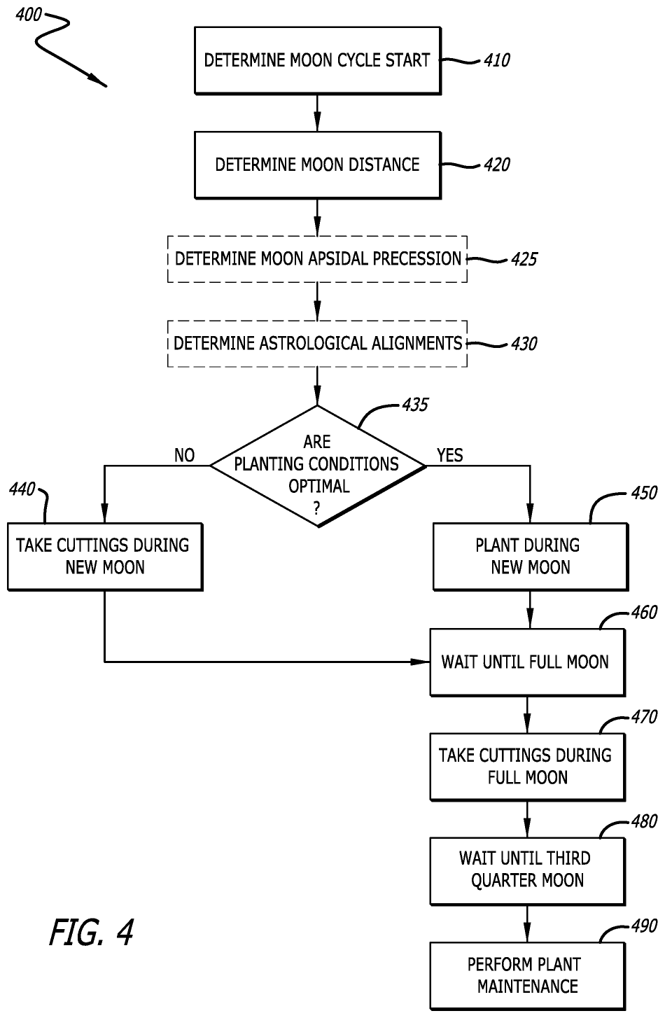

Referring to FIG. 4, a process 400 for determining an optimal time to plant and process CBD-producing plants in accordance with various embodiments of the disclosure is shown. One or more producers of CBD containing products will need to plant, maintain, and cultivate CBD-producing plants over a period of time. The optimal planting conditions are desired to generate increased yields and overall more pleasing and valuable products produced from the plants. Determining the optimal time to plant and maintain the plants can be done by analyzing multiple states of the moon and other astrological bodies.

In many embodiments, the process 400 can determine the date of the moon cycle start (block 410). As discussed above, the moon cycle can start approximately every 29.5 days. Following the current state of the moon and the last known moon cycle start, for example, can be used to plan when the next moon cycle start begins (block 410). Another aspect of the moon that can be utilized is to determine the overall distance between the moon and the Earth (block 420). The moon oscillates between different distances over time. In fact, this distance can be altered by tens of thousands of miles each cycle. The farthest point the moon moves away from Earth is often called the apogee, while the closest point is called the perigee. This oscillation leads to what many refer to as supermoons or micro-moons. While not universally defined, it can be understood that a supermoon occurs when a moon is within ninety percent of the closest perigee distance. Conversely, a micro-moon may be defined as being within ninety percent of the farthest apogee distance. Because of the changes in distance, the effects of the moon on the Earth and the related bodies of water can affect the growing of plants and the selection of an optimal time for growing.

In some embodiments, it may be desired to further examine the moon's apsidal precession state when determining an optimal time for planting (block 425). As those skilled in the art will understand, the moon's orbit does not sit directly parallel with the Earth's orbit. In fact, the moon's orbit is set about five degrees off of Earth's rotation. Furthermore, the moon's orbit is not directly circular and precesses around the Earth approximately every 9 years. As such, the location of where the moon is in relation to the Earth can change over this time as well. Understanding where the apsidal precession is currently can aide in understanding when an optimal time to plant may be.

Similarly, in various embodiments, it may be beneficial to determine the current astrological alignments when making a determination of when to plant (block 430). As discussed above, the moon has a variety of movements that may be tracked in relation to the Earth. Likewise, other astrological bodies that may have some effect on the Earth may also be tracked and analyzed when determining when to plant. Often, by using shorthand or astrological charts such as the ones described within the discussion of FIG. 3, the determination can be made easier based on known data. Once all of these data points have been determined and analyzed both alone and together within one or more combinations and/or weights, the determination over when or what conditions are optimal for planting CBD-producing products can be made (block 435).

If it is decided that the next moon cycle start is not optimal for planting, then one or more producers may simply take cuttings during the new moon from previously budding plants (block 440). The producers can then wait until the full moon within the current lunar cycle to make the next major steps (block 460). However, if it is determined the next moon cycle start will present optimal conditions for planting, one or more producers can plant a plurality of CBD-producing plants during the new moon (block 450). Upon finishing the planting, these producers may also wait until a next full moon cycle (block 460).

Although discussion of the planting, cultivation, and/or harvesting of CBD-producing plants is discussed herein within the scope of lunar cycles, it should be apparent that most, if not all, CBD-producing plants will require more than one lunar cycle to be ready for harvesting, and may require a half-dozen or more cycles until maturity occurs. However, as with most farming, producers can work across multiple fields and multiple gardens that may be in various stages of development. It should be recognized that while discussion within the figures presented herein is presented within one cycle, the actions taken do not indicate that it all must be done in one cycle or that only one crop, garden, or other partition of plants must be processed at a time. In fact, producers may cultivate any number of plants in any number of stages of development.

With that understanding, the producers may wait until a full moon cycle (which may be many cycles later) before taking more actions beyond normal watering and care (block 460). Upon arrival of a full moon, the producer may take cuttings from one or more plants that are suitable for harvesting (block 470). As those skilled in the art will understand, the process of cuttings can occur on any perceived bud of the plant that appears to be ready/ripe, etc. The producers may then wait until a third quarter moon to take actions beyond normal watering/care of the plants (block 480). In the process 400 depicted in FIG. 4, the producers may perform plant maintenance during the third quarter moon (block 490). This maintenance may include trimming, moving, providing growing supplements, and/or pest removal steps. Any care of the plants that is not done on a daily, or semi-regular basis may be done during this period as the effects on the plants may be conducive to these actions.

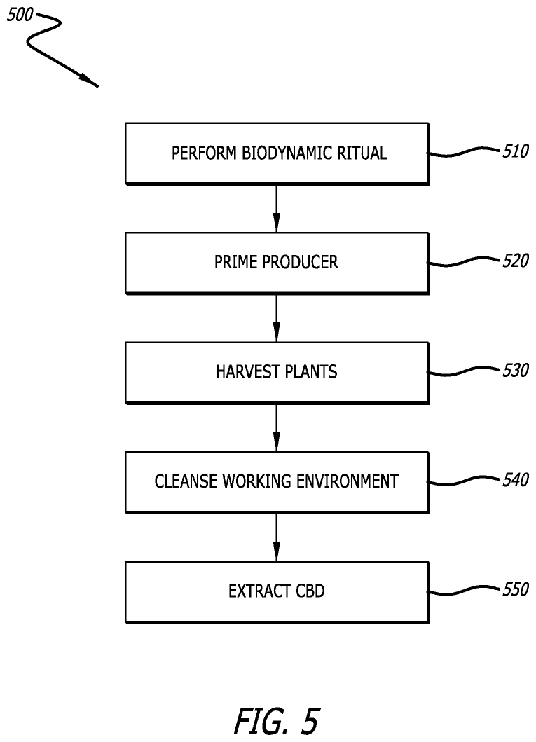

Referring to FIG. 5, a process 500 for producing CBD containing products in accordance with various embodiments of the disclosure is shown. The general process for harvesting CBD-producing plants can include a number of steps that are followed for various reasons. For example, a biodynamic ritual may be performed to begin the process 500 (block 510). In certain cases, the biodynamic ritual may be to prepare the workspace for the upcoming process in the form of a secular ritual so as to create a uniform setting for working.

An additional part of the process 500 may include priming the producers before harvesting (block 520). Because the act of harvesting is based on a series of personal choices in regards to what plants/buds are ripe and ready to take, priming the producer to make better choices is often desired. In some embodiments, the producer may be presented with a plurality of olfactory stimuli that will aide in the selection of plants for harvesting. For example, a selection of optimal, or previously harvested plants/buds may be presented for smelling in order to prime the mind of the producer of what to look for before harvesting. In further embodiments, one or more airborne chemicals may be introduced into the workspace, which may include, for example, the burning of one or more items to produce smoke.

Priming the producer may also come in the form of one or more meditations. These meditations may also be ritualistic in practice and shared among multiple producers within the same workspace, in order to facilitate enhanced and uniform performance between all of the producers. Meditation may also be formatted to allow the producers to focus on the upcoming task, the moon and astrological events currently happening, and/or other event such as previously harvested plants/bud and what they looked and smelled like. In certain embodiments, the olfactory stimuli and meditation may be combined into a single event over several steps that primes the producer to excel at the upcoming harvest.

Once primed, the producers may then harvest the plants as they see fit (block 530). Through the combination of planting and harvesting CBD-producing plants at optimal times and priming the producers through biodynamic rituals and/or meditation, the selection of plants for harvesting will be more optimal than through the use of traditional farming methods. In some embodiments, the harvesting can be done at night under moonlight. In additional embodiments, the harvesting may be done in a group setting, thus giving two producers a voice in selecting plants/buds for harvesting.

Once harvested, the producers may cleanse the working environment prior to extraction of the CBD (block 540). Cleansing the working environment can involve traditional cleansing, but may also indicate a mental, spiritual, and/or airborne cleansing. For example, cleansing may include treating one or more of the ingredients prior to their use in extracting CBD from the harvested CBD-producing plants. For instance, the extraction process may use one or more liquid solutions to extract CBD. These solutions may be treated prior to use for increased effectiveness and/or altering composition. In many embodiments, the treatments may include introducing one or more airborne chemicals into the working environment prior to extraction. In more embodiments, the producers may also be exposed to these same airborne chemicals within the workspace. In certain embodiments, the airborne chemicals may be smoke produced from burning one or more plants. As discussed above, a variety of embodiments utilize burning sage plants which can be used for this purpose, however other suitable plants may be utilized as the producers see fit. It can be considered that the cleansing of the working environment may alter, flavor, enhance, or otherwise modify the producers and materials to be utilized in the extraction process. New cleansing products may be found as more desired results are determined.

Upon completion of the cleansing process, one or more of the producers may begin to extract the CBD from the harvested CBD-producing plants (block 550). As discussed above, there is a large variety of means to extract CBD from plant material. In a number of embodiments, one or more liquid solutions (often solvents) are utilized in order to soak the harvested plants. Often, once soaked in the liquid extracting solution, the remaining plant material may be without use and may be discarded.

In many embodiments, solvents are utilized to separate the CBD molecules from other compounds within the CBD-producing plants. Once separated, the solvent can be configured to quickly evaporate, leaving behind refined CBD oil that can be utilized in one or more products. In further embodiments, the extraction process can utilize a pressurized (i.e., supercritical) carbon dioxide method that pressurizes the gas until it forms into a liquid. This pressurized liquid can then be mixed within a chamber that contains the harvested CBD-producing plants. This pressurized carbon dioxide liquid can then be used to separate the CBD from the plants. The resulting mix of carbon dioxide liquid and CBD oils may be guided to another chamber that releases the pressure, turning the carbon dioxide back into a gas, leaving the extracted CBD oil by itself for further use. Further refining may be needed when using these methods prior to use in one or more CBD containing products.

The use of alcohol extraction methods may also be utilized. By selecting ethanol, butane, or isopropyl alcohol, extraction of CBD may be simpler to achieve. One concern when doing the extraction process is to maintain the natural chemical properties of the CBD-producing plant during extraction. Another concern is the resultant taste that may occur due to various chemicals not fully evaporating during the extraction process. Thus, various methods of performing the extraction process can be altered in order to better enhance the resultant properties of the extracted CBD oil.

In certain embodiments, the act of cleansing the work environment may alter the chemical composition of the work materials to positively affect the resultant CBD extraction compounds as well as the taste of the CBD oil once utilized in one or more CBD containing products. For example, by burning one or more plants and waving the smoke over the instruments and ingredients used for extraction, the smoke may become infused with the materials such that the final taste of the CBD oil is changed for the better. The selection of which cleansing materials to use may differ based on the desired CBD containing products that will be made from the extracted CBD oil. Treating one or more ingredients prior to use can directly affect the chemical composition of the final product.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. To the extent there are variations of the invention, which are within the spirit of the disclosure or equivalent to the inventions found in the claims, it is the intent that this patent will cover those variations as well. Therefore, the present disclosure is to be understood as not limited by the specific embodiments described herein, but only by scope of the appended claims.

What is claimed is:

1. A method for producing CBD-containing products, comprising:
determining an optimal time for planting new CBD-producing plants by:
determining an instance of a new moon; and
determining the current distance from the moon from the Earth;
planting CBD-producing plants during the determined optimal time;
maintaining the CBD-producing plants during third quarter moon lunar cycles;
priming a producer for harvesting through one or more biodynamic rituals;
harvesting at least a portion of the CBD-producing plants at nighttime during a full moon lunar cycle;
cleansing the working environment upon completion of harvesting; and
extracting CBD from the CBD-producing plants directly upon completion of the cleansing
adding the extracted CBD to one or more products.

2. The method of claim 1, wherein priming the producer includes introducing one or more olfactory stimuli that will aide in the selection of plants for harvesting.

3. The method of claim 1, wherein priming the producer includes performing one or more meditations prior to harvesting.

4. The method of claim 1, wherein cleansing further includes treating one or more ingredients used in the extracting process.

5. The method of claim 4, wherein treating includes introducing one or more airborne chemicals into the working environment.

6. The method of claim 5, wherein the producers are also exposed to the one or more airborne chemicals prior to extraction.

7. The method of claim 6, wherein the airborne chemical is smoke produced by burning one or more plants.

8. The method of claim 1, wherein the determination of an optimal time for planting new CBD-producing plants further includes:
determining one or more astrological alignments; and
determining the state of the Moon's apsidal precession.

9. The method of claim 8, wherein the astrological alignments are determined through the use of a zodiac chart.

10. The method of claim 8, wherein a close apsidal precession indicates a more optimal time to plant CBD-producing plants.

11. The method of claim 1, wherein the determination of the moon's distance from the Earth indicates an optimal time to plant CBD-producing plants when the moon is classified as a supermoon.

12. The method of claim 11, wherein the supermoon is defined as the moon being within ninety percent of maximum apogee.

13. The method of claim 1, wherein the determination of the moon's distance from the Earth indicates a non-optimal time to plant CBD-producing plants when the moon is classified as a micro-moon.

14. The method of claim 13, wherein the micro-moon is defined as the moon being within ninety percent of maximum perigee.

15. The method of claim 1, wherein the determination of an optimal time to plant CBD-producing plants may be reversed when operating within the southern hemisphere of the Earth.

16. The method of claim 1, wherein maintaining CBD-producing plants includes removing pests and providing growing supplements.

17. The method of claim 1, wherein extracting comprises infusing the cannabis with olive oil followed by a straining of any plant matter to produce a CBD concentrate.

18. The method of claim 1, wherein the cleansing comprises subjecting the cannabis to carbon dioxide gas under high pressure and extracting comprises straining any plant matter under very low temperature to produce a CBD concentrate.

19. The method of claim 17, wherein the CBD concentrate is added to a product configured to be edible by a person so as to ingest the CBD.

20. A method for producing CBD-containing products, comprising:
determining an optimal time for planting new CBD-producing plants by:
determining an instance of a new moon;
determining the current distance from the moon from the Earth;
determining one or more astrological alignments by referencing a zodiac sign; and
determining the state of the Moon's apsidal precession.
planting CBD-producing plants during the determined optimal time;
maintaining the CBD-producing plants during one or more third quarter moon lunar cycles according to the current zodiac sign;
prior to harvesting, priming a producer for harvesting through one or more biodynamic rituals that aide in the harvesting of CBD-producing plants;
harvesting at least a portion of the CBD-producing plants at nighttime during a full moon lunar cycle;
cleansing the working environment and harvested plants upon completion of harvesting;
extracting CBD from the CBD-producing plants directly upon completion of the cleansing; and
adding the extracted CBD to one or more products for human consumption.
